(19) United States
(12) Patent Application Publication
(10) Pub. No.: US 2025/0344949 A1
(43) Pub. Date: Nov. 13, 2025

(54) Method for Stimulating and Quantifying Physiological Response of Retinal Cells Using Optical Imaging

(71) Applicants: University of Washington, Seattle, WA (US); The Board of Trustees of the Leland Stanford Junior University, Stanford, CA (US)

(72) Inventors: Ramkumar Sabesan, Seattle, WA (US); Vimal Prabhu Pandiyan, Seattle, WA (US); Daniel Palanker, Seattle, WA (US)

(21) Appl. No.: 19/276,976

(22) Filed: Jul. 22, 2025

Related U.S. Application Data
(63) Continuation-in-part of application No. 17/605,182, filed on Oct. 20, 2021.
(60) Provisional application No. 62/839,072, filed on Apr. 26, 2019.

(51) Int. Cl. A61B 3/14; G16H 30/40

(52) U.S. Cl. CPC A61B 3/14; G16H 30/40

(57) ABSTRACT

A method includes capturing one or more first images of retinal cells of an eye and illuminating the retinal cells with a first light after capturing the one or more first images, to cause the retinal cells to exhibit a first response. The method also includes capturing one or more second images of the retinal cells after illuminating the retinal cells with the first light and illuminating the retinal cells with a second light after capturing the one or more second images, to cause the retinal cells to exhibit a second response. The method also includes capturing one or more third images of the retinal cells after illuminating the retinal cells with the second light. The method also includes generating an output, using the first images, the second images, and the third images. The output quantifies the first response and the second response.

### CROSS-REFERENCE TO RELATED APPLICATION

This application is a continuation-in-part of U.S. application Ser. No. 17/605,182, filed on Apr. 25, 2020, which is a § 371 national stage of international application no. PCT/US2020/029984, filed on Apr. 25, 2020, which claims priority to U.S. provisional application No. 62/839,072, filed on Apr. 26, 2019, the entire contents of all of which are incorporated by reference herein.

### BACKGROUND

Retinal diseases are a leading cause of blindness and other vision disorders. To identify and treat retinal diseases, methods capable of imaging both the structure of the retina and the retina's response to visual stimuli are useful. Both high spatial resolution and high temporal resolution are important for obtaining useful information about the retina. Conventional techniques for imaging the structure and/or response of the retina are often lacking in high spatial resolution, high temporal resolution, and/or good signal to noise ratio.

### SUMMARY

A first example is a method comprising: capturing one or more first images of retinal cells of an eye; illuminating the retinal cells with a first light after capturing the one or more first images, to cause the retinal cells to exhibit a first physiological response; capturing one or more second images of the retinal cells after illuminating the retinal cells with the first light; illuminating the retinal cells with a second light after capturing the one or more second images, to cause the retinal cells to exhibit a second physiological response; capturing one or more third images of the retinal cells after illuminating the retinal cells with the second light; and generating an output, using the one or more first images, the one or more second images, and the one or more third images, wherein the output quantifies the first physiological response and the second physiological response.

A second example is a non-transitory computer readable medium storing instructions that, when executed by an imaging device, cause the imaging device to perform the method of the first example.

A third example is an imaging device comprising: one or more processors; an image sensor; a light source; a user interface; and a computer readable medium storing instructions that, when executed by the one or more processors, cause the imaging device to perform the method of the first example.

When the term “substantially” or “about” is used herein, it is meant that the recited characteristic, parameter, or value need not be achieved exactly, but that deviations or variations, including, for example, tolerances, measurement error, measurement accuracy limitations, and other factors known to those of skill in the art may occur in amounts that do not preclude the effect the characteristic was intended to provide. In some examples disclosed herein, “substantially” or “about” means within +/−0-5% of the recited value.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate by way of example only and, as such, that numerous variations are possible.

### BRIEF DESCRIPTION OF THE DRAWINGS

FIG. 1 is a schematic diagram of an optical instrument, according to an example.

FIG. 2 is a block diagram of a computing system, according to an example.

FIG. 3 is a schematic diagram of captured images, according to an example.

FIG. 4 is a schematic diagram of transformed images, according to an example.

FIG. 5 is schematic diagram of imaging techniques, according to an example.

FIG. 6 is a block diagram of a method, according to an example.

FIG. 7 is a block diagram of an imaging device, according to an example.

FIG. 8 is a schematic diagram of a light source, an image sensor, and an eye, according to an example.

FIG. 9 shows images captured by an image sensor, according to an example.

FIG. 10 is a graph depicting a change in optical path length with respect to time for one or more retinal cells, according to an example.

FIG. 11 is a block diagram of a method, according to an example.

FIG. 12A is an image depicting optical phase change of an area of a retina, according to an example.

FIG. 12B is a graph depicting components of a change in optical path length with respect to time for one or more retinal cells, according to an example.

FIG. 13 shows timing diagrams for methods of illuminating retinal cells, according to an example.

### DETAILED DESCRIPTION

Retinal photoreceptor cells facilitate vision by converting incident photons to electrical activity. High acuity spatial vision, color vision, and light adaptation, which are hallmarks of normal everyday visual function, are all facilitated by cone photoreceptors. Thus, loss or dysfunction of cones due to age-related or inherited retinal disease is debilitating and diminishes quality of life. Therapies in development aim to repair or regenerate photoreceptors afflicted by disease and to thereby restore vision. Realizing the potential of such therapies can be aided by establishing baseline physiological responses of the cones against which the efficacy of the treatment can be compared, ideally in living human eyes at cellular resolution. Techniques described herein are useful for the non-invasive assessment of normal cone function, disease progression, and treatments at high spatiotemporal resolution in humans. These techniques can be used to measure shape changes or refractive index changes of individual human cone cells at a nanometer-millisecond scale in response to the cones being excited by light.

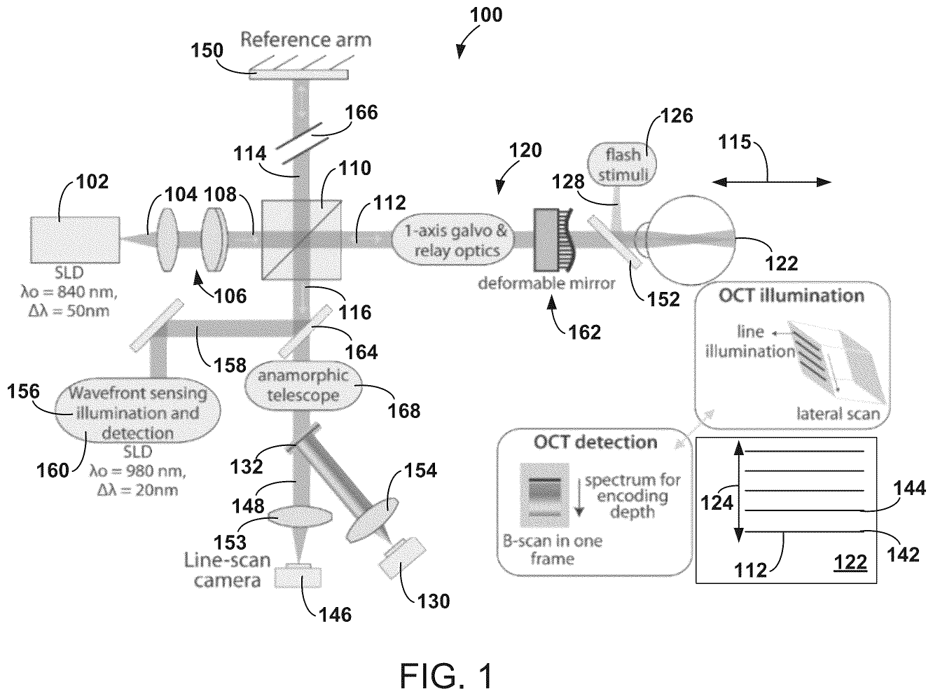

FIG. 1 is a schematic diagram of an optical instrument 100. The optical instrument 100 includes a first light source 102 configured to generate a broadband light 104 and an optical module 106 configured to collimate the broadband light 104 and focus the broadband light 104 into a line 108 (e.g., having a length ranging from 400 μm to 500 μm on the retina 122). The optical instrument 100 also includes a beam splitter 110 configured to split the broadband light 104 into a sample beam 112 and a reference beam 114 and configured to combine the reference beam 114 with the sample beam 112 to form an interference beam 116. The optical instrument 100 also includes a control system 120 configured to scan the sample beam 112 on the retina 122 of a subject along an axis 124 that is substantially perpendicular to the sample beam 112. The optical instrument 100 also includes a second light source 126 configured to stimulate the retina 122 with a visible light 128 to induce a physical change within the retina 122 such that the sample beam 112 is altered by the physical change. The optical instrument 100 also includes an image sensor 130 and a dispersive element 132 configured to receive the interference beam 116 from the beam splitter 110 and to disperse the interference beam 116 onto the image sensor 130.

The optical instrument 100 and the recorded light-induced optical changes from the retina 122 can be referred to as an optoretinogram.

The first light source 102 can include a super-luminescent diode or a supercontinuum source, but other examples are possible.

The broadband light 104 can have a center wavelength of 840 nanometers (nm) and/or a full width half maximum (FWHM) within a range of 15 nm to 150 nm, (e.g., 50 nm). When leaving the first light source 102, the broadband light 104 is generally not collimated or focused.

The optical module 106 includes a positive powered lens or a mirror that collimates the broadband light 104 and a cylindrical lens that focuses the broadband light into the line 108. Other examples are possible.

The beam splitter 110 generally takes the form of two triangular prisms that are adhered to each other to form a cube, or a plate beam splitter. The discontinuity between the two prisms performs the beam splitting function. Thus, the beam splitter 110 splits the line-shaped broadband light 104 into the sample beam 112 and the reference beam 114. The reference beam 114 travels from the beam splitter 110, through the optical module 166, reflects off the mirror 150, travels back through the optical module 166, and back to the beam splitter 110. The sample beam 112 is scanned by the control system 120 and/or formed by the deformable mirror 162, and transmits through the filter 152 onto the retina 122. The sample beam 112 reflects and/or scatters off of the retina 122, travels through the filter 152, and back to the beam splitter 110. The beam splitter 110 combines the reference beam 114 with the sample beam 112 to form the interference beam 116. Thus, the interference beam 116 constitutes a superposition of the reference beam 114 and the sample beam 112, and the optical instrument 100 can operate as an interferometer.

The optical module 166 is configured to maintain collimation and/or coherence of the reference beam 114. The distance between the beam splitter 110 and the mirror 150 can be several meters are more, and the collimation and/or coherence of the reference beam 114 can be degraded over such distances without compensation. Thus, the optical module 166 can include lenses and/or mirror-based telescopes that maintain collimation and/or coherence of the reference beam 114.

The mirror 150 is configured to reflect the reference beam 114 back to the beam splitter 110. The mirror 150 generally has a reflectance that is substantially equal to 100% over the visible and infrared spectrum, but other examples are possible.

The control system 120 can include a galvanometer that can scan (e.g., deflect) the sample beam 112 along an axis 124 on the retina 122 (inset at the bottom right of FIG. 1). As shown, the axis 124 is perpendicular to the sample beam 112. For example, the control system 120 can scan the sample beam 112 such that the sample beam 112 illuminates a line-shaped position 142 on the retina 122, and then illuminates a line-shaped position 144 on the retina 122, and so on. The control system 120 can also control the deformable mirror 162, as described in more detail below. The control system 120 generally includes hardware and/or software configured to facilitate performance of the functions attributed to the control system 120 herein.

The sample beam arm of the optical instrument 100 can also include an optical module similar to the optical module 166 that is configured to maintain collimation and/or coherence of the sample beam 112 (referred to as “relay optics” in FIG. 1).

The second light source 126 can take the form of a light emitting diode, but other examples are possible. The visible light 128 can have a full width half maximum (FWHM) within a range of 10 nm to 50 nm and have a center wavelength of 528 nm, 660 nm, or 470 nm, for example. The visible light 128 could generally have any center wavelength within the visible light spectrum. The visible light 128 is directed upon the retina 122 by the filter 152. The visible light 128 can induce physical changes in the retina 122 such as movement and/or changes in size or shape of retinal neurons in any of the three dimensions. In some examples, the physical change in the retina 122 can include a change in refractive index and/or optical path length of one or more retinal neurons, a change in electrical activity in one or more retinal neurons, and/or a change in constituents of one or more retinal neurons. In some examples, the visible light 128 consists of one or more pulses of light having varying or constant pulse widths (e.g., 500 us to 100 ms) and/or intensities, but other examples are possible.

The filter 152 is configured to direct the visible light 128 to the retina 122 and to transmit the sample beam 112 from the retina 122 back to the beam splitter 110. Thus, the filter 152 has a non-zero transmissivity for at least infrared light.

The image sensor 130 typically takes the form of a complementary metal-oxide-semiconductor (CMOS) or charge-coupled device (CCD) image sensor (e.g., a high speed camera).

The dispersive element 132 is typically a diffraction grating (e.g., transmissive or reflective), but a prism could be used as well. Other examples are possible. The dispersive element 132 is configured to receive the interference beam 116 from the beam splitter 110 (e.g., from the optical module 164) and to diffract the interference beam 116 onto the image sensor 130. That is, dispersive element 132 disperses the interference beam 116 such that varying spectral components of the interference beam 116 are distinguishable (e.g., positioned on respective lines/portions of the image sensor 130).

The image sensor 146 (e.g., a line scan camera) is configured to capture a substantially one-dimensional image representing a zero-order portion 148 of the interference beam 116 that passes through the dispersive element 132 without being diffracted. When the image sensor 146 is being operated, the reference beam 114 is blocked from the beam splitter 110. Thus, in this example, the interference beam 116 is substantially the same as the sample beam 112 that returns from the retina 122. The zero-order portion 148 of the interference beam 116 is a signal that represents a portion of the sample beam 112 that is back-scattered from the retina 122. The one-dimensional image represents a line-shaped portion of a surface of the retina 122 that is illuminated by the sample beam 112 (e.g., the portion of the retina 122 at position 142). The image sensor 146 can capture one-dimensional images corresponding respectively to various positions on the retina 122 along the axis 124, for example. These one-dimensional images can be pieced together to form a two-dimensional image representing an exposed surface of the retina 122 (e.g, before, during, and/or after stimulation by the visible light 128).

The optical module 153 is configured to adjust a spatial resolution of the zero-order portion 148 and/or focus the zero-order portion 148 so that the area of the image sensor 146 can be efficiently used. The optical module 153 can include one or more lenses and/or mirrors.

The optical module 154 is configured to modify the interference beam 116 after the interference beam 116 has been dispersed by the dispersive element 132 to adjust spatial resolution of the interference beam 116 and/or adjust spectral resolution of the interference beam 116 so that the area of the image sensor 130 can be efficiently used. The optical module 154 can include one or more lenses and/or mirrors and can also be used to focus the interference beam 116 after the interference beam 116 has been dispersed by the dispersive element 132.

The optical module 168 (e.g., an anamorphic telescope), including one or more lenses and/or mirrors, is configured to compress or stretch the interference beam 116 before the interference beam 116 has been dispersed by the dispersive element 132. The optical module 168 typically will include two cylindrical lenses having longitudinal axes that are parallel to each other but rotated at 90 degrees with respect to each other.

The optical instrument 100 also includes a third light source 156 configured to generate a third light 158. The third light source 156 could be an LED, but other examples are possible. The third light 158 can have a center wavelength of 970 nm and a FWHM of 10-30 nm (e.g., 20 nm), but other examples are possible. The optical instrument 100 also includes a wavefront sensor 160 and a second optical module 164 including one or more mirrors and/or lenses configured to direct the third light 158 from the third light source 156 to the beam splitter 110 and from the beam splitter 110 back to the wavefront sensor 160. The beam splitter 110 is further configured to direct the third light 158 to the control system 120. The wavefront sensor 160 is configured to detect optical aberrations of an eye of the subject by analyzing the third light 158 that returns from the retina 122. The control system 120 is configured to control the deformable mirror 162 to form the sample beam 112 on the retina 122 based on the optical aberrations of the eye, (e.g., to compensate for the aberrations of the eye).

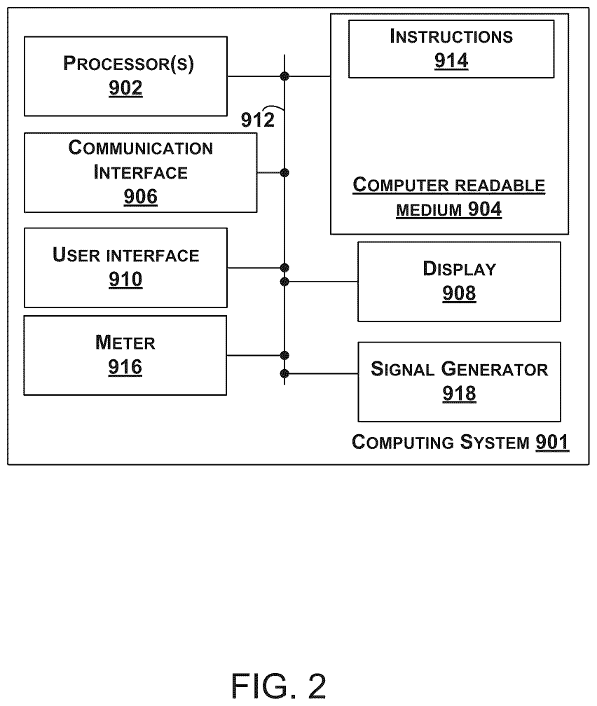

FIG. 2 shows the computing system 901. The computing system 901 includes one or more processors 902, a non-transitory computer readable medium 904, a communication interface 906, a display 908, and a user interface 910. Components of the computing system 901 are linked together by a system bus, network, or other connection mechanism 912.

The one or more processors 902 can be any type of processor(s), such as a microprocessor, a digital signal processor, a multicore processor, etc., coupled to the non-transitory computer readable medium 904.

The non-transitory computer readable medium 904 can be any type of memory, such as volatile memory like random access memory (RAM), dynamic random access memory (DRAM), static random access memory (SRAM), or non-volatile memory like read-only memory (ROM), flash memory, magnetic or optical disks, or compact-disc read-only memory (CD-ROM), among other devices used to store data or programs on a temporary or permanent basis.

Additionally, the non-transitory computer readable medium 904 can be configured to store instructions 914. The instructions 914 are executable by the one or more processors 902 to cause the computing system 901 to perform any of the functions or methods described herein.

The communication interface 906 can include hardware to enable communication within the computing system 901 and/or between the computing system 901 and one or more other devices. The hardware can include transmitters, receivers, and antennas, for example. The communication interface 906 can be configured to facilitate communication with one or more other devices, in accordance with one or more wired or wireless communication protocols. For example, the communication interface 906 can be configured to facilitate wireless data communication for the computing system 901 according to one or more wireless communication standards, such as one or more Institute of Electrical and Electronics Engineers (IEEE) 801.11 standards, ZigBee standards, Bluetooth standards, etc. As another example, the communication interface 906 can be configured to facilitate wired data communication with one or more other devices.

The display 908 can be any type of display component configured to display data. As one example, the display 908 can include a touchscreen display. As another example, the display 908 can include a flat-panel display, such as a liquid-crystal display (LCD) or a light-emitting diode (LED) display.

The user interface 910 can include one or more pieces of hardware used to provide data and control signals to the computing system 901. For instance, the user interface 910 can include a mouse or a pointing device, a keyboard or a keypad, a microphone, a touchpad, or a touchscreen, among other possible types of user input devices. Generally, the user interface 910 can enable an operator to interact with a graphical user interface (GUI) provided by the computing system 901 (e.g., displayed by the display 908).

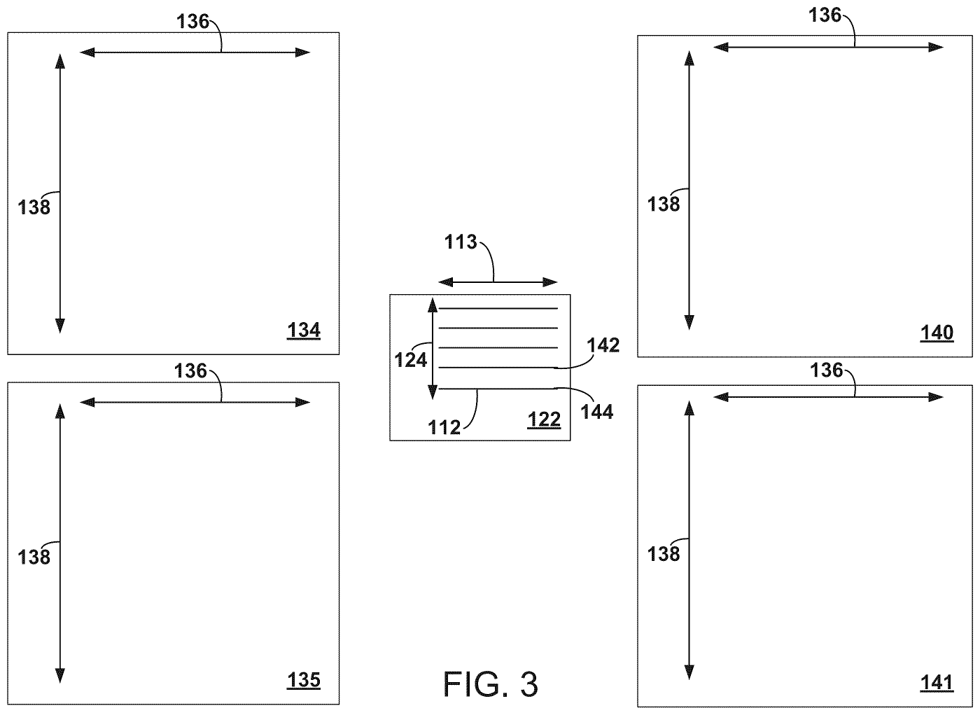

FIG. 3 is a schematic diagram of captured images 134, 135, 140, and 141.

The image sensor 130 is configured to capture a wavelength space image 134 of the interference beam 116 after the interference beam 116 has been dispersed by the dispersive element 132. The wavelength space image 134 is defined by an axis 136 that corresponds to a length 113 of the sample beam 112 and an axis 138 that corresponds to wavelengths of the sample beam 112. That is, wavelengths of the interference beam 116 are dispersed along the axis 138 in order of increasing or decreasing wavelength. The wavelength space image 134 corresponds to the position 142 on the retina 122 along the axis 124. Thus, the wavelength space image 134 corresponds to a cross section or “slice” of the retina 122 corresponding to a plane defined by the sample beam 112 at the position 142 being extended into the retina 122, with the varying wavelengths of the interference beam 116 being a proxy for a depth 115 into the retina 122, as explained further below.

The image sensor 130 is also configured to capture a wavelength space image 140 of the interference beam 116 after the interference beam 116 has been dispersed by the dispersive element 132. The wavelength space image 140 is also defined by the axis 136 and the axis 138. Similar to the wavelength space image 134, in the wavelength space image 140, wavelengths of the interference beam 116 are dispersed along the axis 138 in order of increasing or decreasing wavelength. The wavelength space image 140 corresponds to the position 144 on the retina 122 along the axis 124. Thus, the wavelength space image 140 corresponds to a cross section or “slice” of the retina 122 corresponding to a plane defined by the sample beam 112 at the position 144 being extended into the retina 122.

In some embodiments, the image sensor 130 captures additional wavelength space images 135 and 141 subsequent to capturing the wavelength space images 134 and 140 and/or after the retina 122 is stimulated with the visible light 128. In this context, the image sensor 130 can capture the wavelength space image 135 of the interference beam 116 after the interference beam 116 has been dispersed by the dispersive element 132. The wavelength space image 135 is also defined by the axis 136 and the axis 138. The wavelength space image 135 corresponds to the position 142 on the retina 122 along the axis 124. Thus, the wavelength space image 135 corresponds to a cross section or “slice” of the retina 122 corresponding to a plane defined by the sample beam 112 at the position 142 being extended into the retina 122, after the visible light 128 stimulates the retina 122. Thus, the wavelength space image 135 can be compared to the wavelength space image 134 to determine an effect of the visible light 128 at the position 142.

The image sensor 130 can also capture the wavelength space image 141 of the interference beam 116 after the interference beam 116 has been dispersed by the dispersive element 132. The wavelength space image 141 is also defined by the axis 136 and the axis 138. The wavelength space image 141 corresponds to the position 144 on the retina 122 along the axis 124. Thus, the wavelength space image 141 corresponds to a cross section or “slice” of the retina 122 corresponding to a plane defined by the sample beam 112 at the position 144 being extended into the retina 122, after the visible light 128 stimulates the retina 122. Thus, the wavelength space image 141 can be compared to the wavelength space image 140 to determine an effect of the visible light 128 at the position 144.

In some embodiments, the sample beam 112 remains at the position 142 while image data is captured over time. For example, the wavelength space image 135 can be captured (e.g., immediately) after the wavelength space image 134 is captured without scanning the sample beam 112 between capture of the wavelength space image 134 and capture of the wavelength space image 135. This can allow for high temporal resolution scans of one particular cross-sectional area of the retina 122. Such wavelength space images can be transformed into corresponding depth space images that depict signal intensity or signal phase as well, as described below. This technique can also be applied to volumetric scans.

In additional embodiments, the computing system 901 can transform the wavelength space images 134, 140, 135, and 141 into depth space images, as described below.

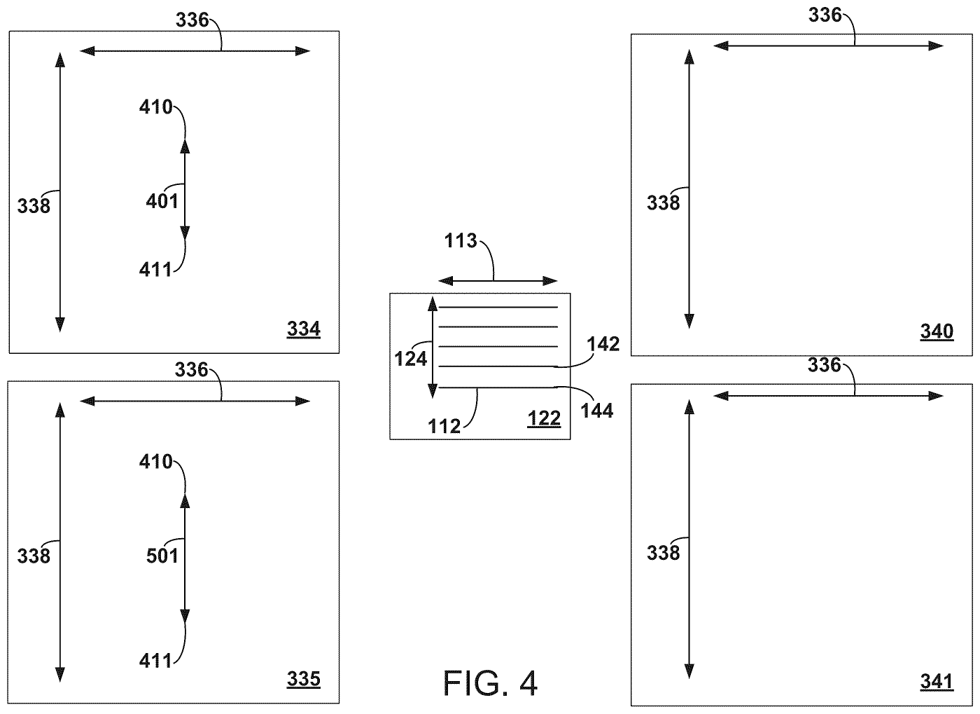

Referring to FIGS. 3 and 4, the computing system 901 can transform the wavelength space image 134 to generate a depth space image 334 comprising a first plurality of pixel values. For example, the computing system 901 can perform a Fourier transform that maps the wavelength space to a depth space, the depth space referring to a depth 115 within the retina 122. The depth space image 334 is defined by an axis 336 corresponding to the length 113 of the sample beam 112 and an axis 338 corresponding to the depth 115 into the retina 122. Each pixel value of the first plurality of pixel values indicates an intensity at a particular depth 115 within the retina 122 and at a particular lateral position along the length 113. The depth space image 334 corresponds to the position 142 on the retina 122 along the axis 124.

The computing system 901 can also transform the wavelength space image 140 to generate a depth space image 340 comprising a second plurality of pixel values. The depth space image 340 is defined by the axis 336 and the axis 338. Each pixel value of the second plurality of pixel values indicates an intensity at a particular depth 115 within the retina 122 and a particular lateral position along the length 113. The depth space image 340 corresponds to the position 144 on the retina 122 along the axis 124.

The computing system 901 can also transform the wavelength space image 135 to generate a depth space image 335 comprising a third plurality of pixel values. The depth space image 335 is defined by the axis 336 and the axis 338. Each pixel value of the third plurality of pixel values indicates an intensity at a particular depth 115 within the retina 122 and a particular lateral position along the length 113. The depth space image 335 corresponds to the position 142 on the retina 122 along the axis 124.

The computing system 901 can also transform the wavelength space image 141 to generate a depth space image 341 comprising a fourth plurality of pixel values. The depth space image 341 is defined by the axis 336 and the axis 338. Each pixel value of the fourth plurality of pixel values indicates an intensity at a particular depth 115 within the retina 122 and a particular lateral position along the length 113. The depth space image 341 corresponds to the position 144 on the retina 122 along the axis 124. Thus, wavelength space images can also be used to analyzed the effects that the visible light 128 has on the retina 122.

The computing system 901 is configured to generate a three-dimensional image of the retina 122 by combining the depth space image 334 and the depth space image 340. The computing system 901 is also configured to generate a three-dimensional image of the retina 122 by combining the depth space image 335 and the depth space image 341.

In other embodiments, the wavelength space images 134, 135, 140, and 141 are transformed by the computing system 901 into depth space images 334, 340, 335, and 341 that depict phase of the interference beam 116 corresponding to various positions within the retina 122, instead of intensity of the interference beam 116 corresponding to various positions within the retina 122. The absolute value of the transformed data corresponds to signal intensity of the interference beam 116 whereas the argument of the transformed data corresponds to relative phase of the interference beam 116.

In examples where the depth space images 334, 335, 340, and 341 depict signal phase of the interference beam 116, the computing system 901 can be further configured to use the depth space image 334 to determine a first optical path length 401 that separates a first end 410 of an object (e.g., a retinal neuron) from a second end 411 of the object. Generally, the computing system 901 will use the depth space image 334 to determine a first signal phase difference between the signal phase corresponding to the first end 410 and the signal phase corresponding to the second end 411, and use the first signal phase difference to derive the first optical path length 401. In some examples, the depth space image 334 represents a first time, for example, before the retina 122 is stimulated by the visible light 128. In this context, the first end 410 additionally corresponds to a first intensity peak of a corresponding depth space image representing signal intensity obtained at the first time. The second end 411 additionally corresponds to a second intensity peak of the corresponding depth space image representing signal intensity at the first time. The computing system 901 can also use the depth space image 335 to determine a second optical path length 501 that separates the first end 410 and the second end 411 at a second subsequent time, for example, after the retina 122 is stimulated by the visible light 128. Generally, the computing system 901 will use the depth space image 335 to determine a second signal phase difference between the signal phase corresponding to the first end 410 and the signal phase corresponding to the second end 411, and use the second signal phase difference to derive the second optical path length 501. In this context, the first end 410 additionally corresponds to a third intensity peak of the corresponding depth space image representing signal intensity at the second time. The second end 411 additionally corresponds to a fourth intensity peak of the corresponding depth space image representing signal intensity at the second time. Comparing signal phases in this way can yield very high temporal and spatial resolution when analyzing how the retina reacts to stimuli. In a particular embodiment, the detected change in optical path length of a retinal neuron can represent an actual change in size or shape of the retinal neuron, or a change in physiological composition that changes the optical index of the retinal neuron.

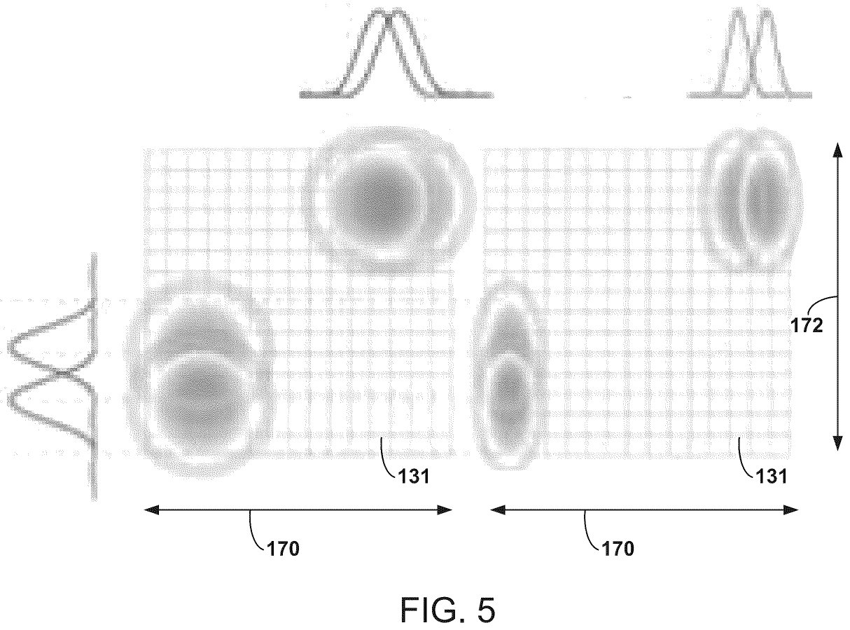

FIG. 5 depicts imaging techniques. For example, the optical module 168 can be used to compress or expand the interference beam 116 independently in the spectral or spatial dimension before the interference beam 116 is dispersed by the dispersive element 132. In a first example, the axis 170 represents the spectral axis of the image sensor 130 and the axis 172 represents the spatial axis of the image sensor 130. Thus, the optical module 168 can be operated to compress the dimension of the interference beam 116 that corresponds to the axis 170 and/or expand the dimension of the interference beam 116 that corresponds to the axis 172, to make efficient use of the area of the image sensor 130. In a second example, the axis 170 represents the spatial axis of the image sensor 130 and the axis 172 represents the spectral axis of the image sensor 130. The ratio of the focal lengths of the cylindrical lenses decides the ratio of the major and minor axes of the ellipses. By reducing the size along the spectrum dimension, better spectral resolution is achievable, without sacrificing spatial resolution along the line dimension.

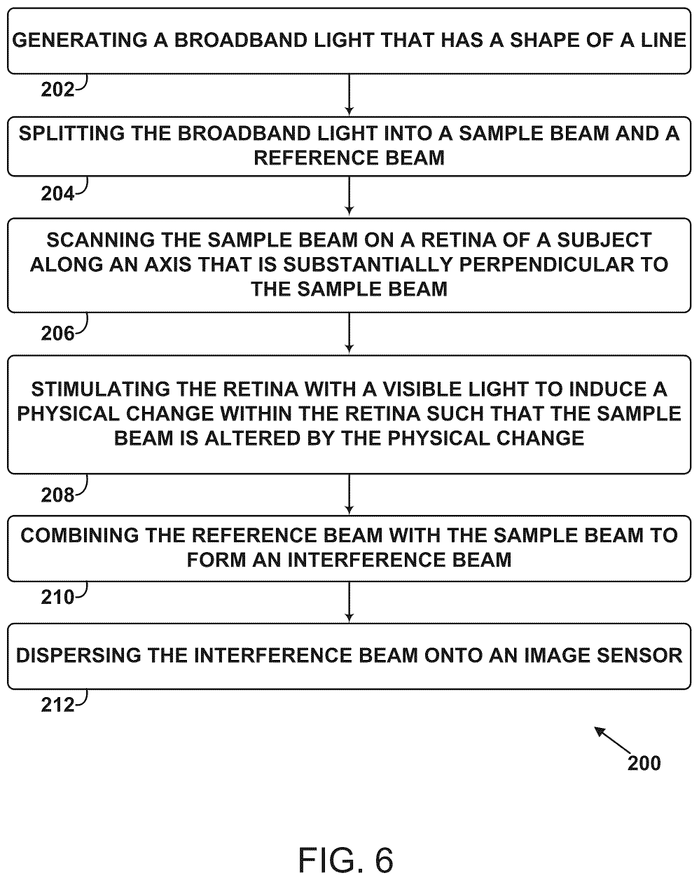

FIG. 6 is a block diagram of a method 200 of operating the optical instrument 100. As shown in FIG. 6, the method 200 includes one or more operations, functions, or actions as illustrated by blocks 202, 204, 206, 208, 210, and 212. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 202, the method 200 includes generating the broadband light 104 that has a shape of the line 108.

At block 204, the method 200 includes splitting the broadband light 104 into the sample beam 112 and the reference beam 114.

At block 206, the method 200 includes scanning the sample beam 112 on the retina 122 of a subject along the axis 124 that is substantially perpendicular to the sample beam 112.

At block 208, the method 200 includes stimulating the retina 122 with the visible light 128 to induce a physical change within the retina 122 such that the sample beam 112 is altered by the physical change.

At block 210, the method 200 includes combining the reference beam 114 with the sample beam 112 to form the interference beam 116.

At block 212, the method 200 includes dispersing the interference beam 116 onto the image sensor 130.

The method 200 can involve non-invasively imaging retinal function in the subject on a cellular scale, detecting a change in size or shape or physiology of a retinal neuron, and/or in-vivo measurement of electrical activity of a/many retinal neuron in the subject. The method 200 can also involve diagnosing a retinal disorder, such as a retinal disorder that affects one or more of photoreceptors, retinal pigment epithelium, choroid, ganglion cells, or a nerve fiber layer, vasculature. The method 200 can also involve determining a physiological composition of a retinal neuron in the subject and determining the change in physiological composition with light stimuli.

The method 200 can also involve treating and/or diagnosing one or more of the following disorders: retinal tear, retinal detachment, diabetic retinopathy, epiretinal membrane, macular hole, wet macular degeneration, dry macular degeneration, retinitis pigmentosa, achromatopsia, and macular telangiectasia.

A retinal tear occurs when the vitreous shrinks and tugs on the retina with enough traction to cause a break in the tissue. A retinal tear is often accompanied by symptoms such as floaters and flashing lights.

Retinal detachment typically occurs in the presence of fluid under the retina. This usually occurs when fluid passes through a retinal tear, causing the retina to lift away from the underlying tissue layers.

Diabetic retinopathy generally involves capillary fluid leakage and/or abnormal capillary development and bleeding into and under the retina, causing the retina to swell, which can blur or distort vision.

Epiretinal membrane generally involves the development of a tissue-like scar or membrane that pulls up on the retina, which distorts vision. Objects may appear blurred or crooked.

Macular hole typically involves a small defect in the center of the retinal macula, which may develop from abnormal traction between the retina and the vitreous, or it may follow an injury to the eye.

Macular degeneration generally involves retinal macula deterioration, causing symptoms such as blurred central vision or a blind spot in the center of the visual field. There are two types-wet macular degeneration and dry macular degeneration. Many people will first have the dry form characterized by the presence of drusen that can distort vision, which can progress to the wet form in one or both eyes, characterized by blood vessel formation under the macula which can bleed and lead to severe vision effects including permanent loss of central vision.

Retinitis pigmentosa is an inherited degenerative disease affecting the retina that causes loss of night and side vision. Retinitis pigmentosa is typically characterized by a breakdown or loss of cells in the retina.

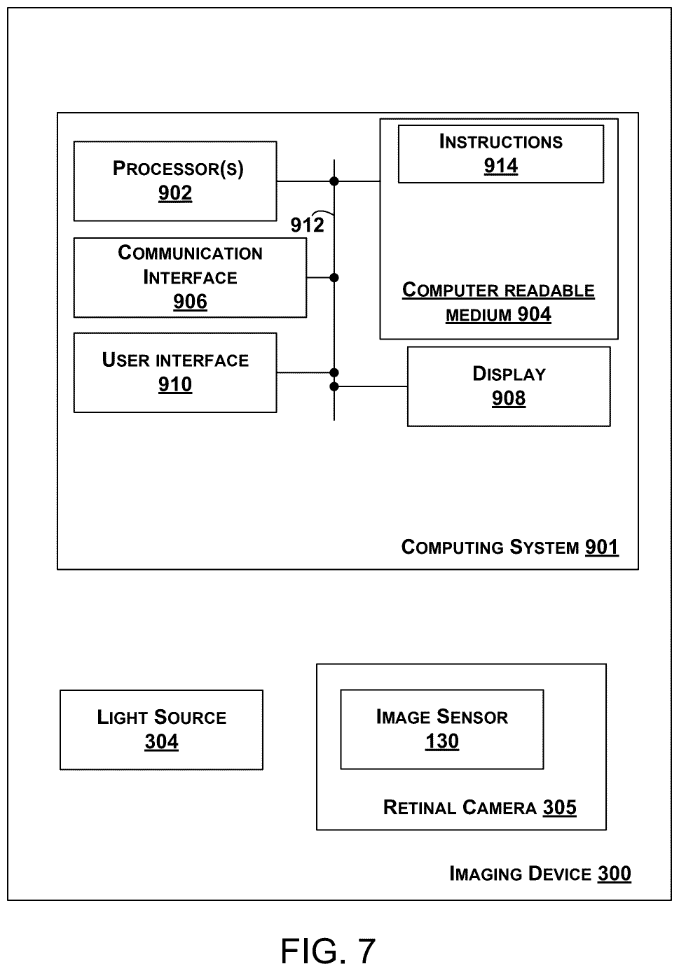

FIG. 7 is a block diagram of an imaging device 300. The imaging device 300 includes the computing system 901, a retinal camera 305 that includes an image sensor 130, and a light source 304. In some examples, the imaging device 300 can include any or all of the components of the optical instrument 100. In some examples, the retinal camera 305 could include its own illumination source distinct from the light source 304 as well as other optical components such as lenses.

The light source 304 can include one or more lasers, light emitting diodes (LEDs), incandescent lamps, halogen lamps, or similar components. The light source 304 is generally configured to generate any combination of intensities and wavelengths (e.g., ultraviolet, visible, or infrared) of light used for illumination and excitation of retinal cells described herein.

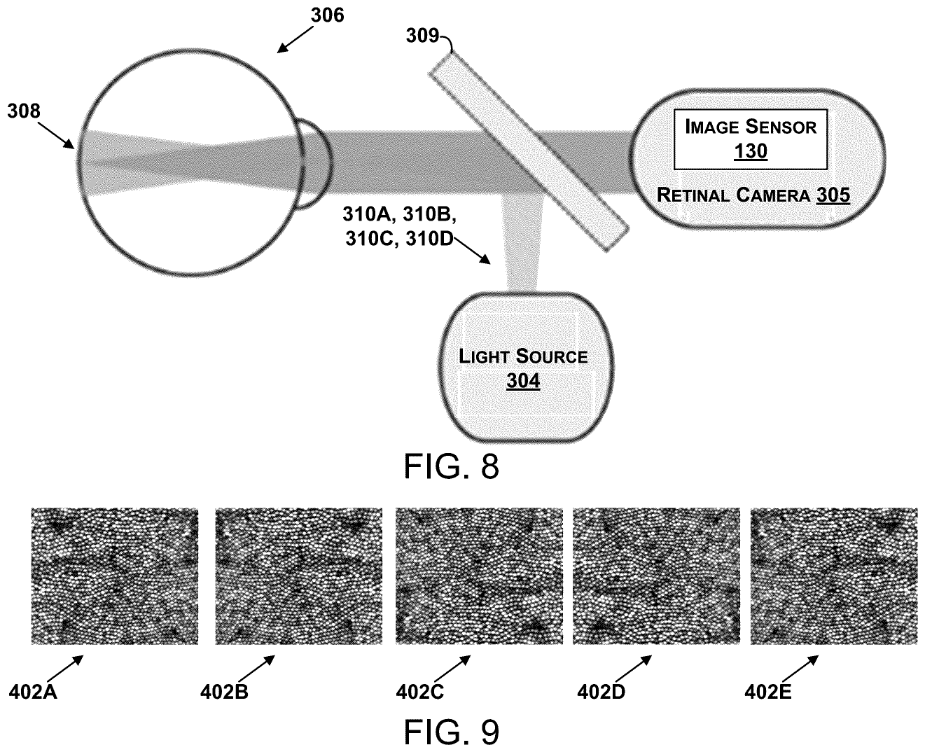

FIG. 8 is a schematic diagram of the light source 304, the retinal camera 305 including the image sensor 130, an eye 306 (e.g., a human eye), and retinal cells 308. As shown, the light source 304 can emit a light 310A, a light 310B, a light 310C, and/or a light 310D. For example, the lights 310A-D can be generated separately and/or sequentially as described below. Some portion of the lights 310A-D are reflected by a reflector 309 (e.g., a beam splitter) onto the retinal cells 308 when each particular light 310 is generated. At least some of the light reflected or otherwise emitted by the retinal cells 308 transmits through the reflector 309 and is captured by the image sensor 130 as described in more detail below.

FIG. 9 shows one or more images 402A, one or more images 402B, one or more images 402C, one or more images 402D, and a composite image 402E. In various examples, the image sensor 130 captures the one or more images 402A, the one or more images 402B, the one or more images 402C, and/or the one or more images 402D using a scanning laser ophthalmoscope, a fundus camera, or optical coherence tomography. Adaptive optics can be used in conjunction with any of the aforementioned optical techniques or with other techniques to capture the one or more images 402A, the one or more images 402B, the one or more images 402C, and/or the one or more images 402D.

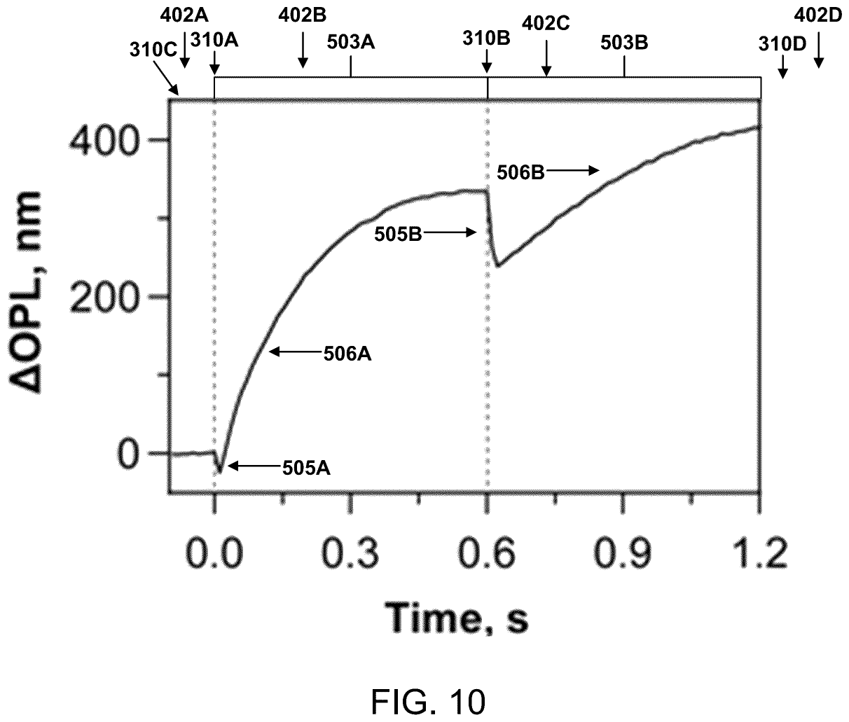

FIG. 10 is a graph depicting change in optical path length in nanometers with respect to time in seconds for one or more of the retinal cells 308.

Referring to FIG. 8 and FIG. 9, the imaging device 300 uses the image sensor 130 to capture the images 402A of the retinal cells 308 of the eye 306. Depending on the imaging technique used, the pixel values of the images 402A, the images 402B, the images 402C, the images 402D, and/or the composite image 402E can represent intensity, color, wavelength, scattering, absorption, fluorescence, and/or phase of the light captured. This retinal back-reflection informs of the physiological processes in the retina-phototransduction, bleaching, structural remodeling, calcium activity, etc.

Generally, a light having wavelengths greater than 780 nanometers (e.g., visible light) can be used to illuminate the retinal cells 308 during any image capture of the retinal cells 308.

The light source 304 illuminates the retinal cells 308 with the light 310A after the image sensor 130 captures the images 402A, to cause the retinal cells 308 to exhibit a physiological response 503A (shown in FIG. 10). In some examples, the image sensor 130 captures the images 402A in periodic succession before the light source 304 illuminates the retinal cells 308 with the light 310A. This is shown chronologically at the top of FIG. 10. In some examples, the light source 304 illuminates the retinal cells 308 with the light 310A with a photon flux density of 10⁴ μm⁻² to 10⁸ μm⁻². Additionally or alternatively, the light source 304 illuminates the retinal cells 308 with the light 310A having one or more pulses (e.g., at least two pulses) with widths greater than 0.01 millisecond and less than 100 milliseconds. In some examples, the light 310A has wavelengths greater than 380 nanometers and less than 780 nanometers.

In some examples, it may be useful to have the retinal cells 308 be acclimated to darkness prior to illuminating the retinal cells 308 with the light 310A. Accordingly, the light source 304 can illuminate the retinal cells 308 with the light 310A after obscuring the retinal cells 308 from light for a time period of 0.1 seconds to 5 minutes, for the purpose of adapting cones to the dark. In this context, the light 310A generally has a photon flux density of at least 105 μm⁻².

In other examples, the light source 304 can illuminate the retinal cells 308 with the light 310A after obscuring the retinal cells 308 from light for a time period of 5 minutes to 30 minutes, for the purpose of adapting rods to the dark. In this context, the light 310A generally has a photon flux density of at least 104 μm⁻².

In some examples, it may be useful to “saturate” the retinal cells 308 with light before illuminating the retinal cells 308 with multiple pulses of the light 310A. Accordingly, the light source 304 illuminates the retinal cells 308 with the light 310C prior to illuminating the retinal cells 308 with the light 310A. In some examples, the light 310C includes wavelengths greater than 380 nanometers and less than 780 nanometers and a photon flux density of at least 104 μm⁻² or at least 105 μm⁻².

As noted above, the illumination of the retinal cells 308 with the light 310A generally causes the retinal cells 308 to exhibit a first physiological response, an example of which is depicted in FIG. 10 as the physiological response 503A. In various examples, the physiological response 503A includes the retinal cells 308 exhibiting a change in shape and/or refractive index. FIG. 10 shows the respective time periods during which changes in shape and/or refractive index occurs, which are both reflected as changes in optical path length. For example, the physiological response 503A includes a contraction 505A during which the optical path length (OPL) of the retinal cells 308 decreases, followed by an expansion 506A during which the OPL increases. The contraction 505A can represent the retinal cells 308 physically shrinking and/or changing their biochemistry such that the refractive index of the retinal cells 308 decreases. The expansion 506A can represent the retinal cells 308 physically expanding and/or changing their biochemistry such that the refractive index of the retinal cells 308 increases. FIG. 10 depicts the respective time periods during which the contraction 505A and the expansion 506A occur and the changes in OPL.

The image sensor 130 also captures the images 402B of the retinal cells 308 after the light source 304 illuminates the retinal cells 308 with the light 310A. In some examples, the image sensor 130 captures the images 402B in periodic succession after the light source 304 illuminates the retinal cells 308 with the light 310A. The images 402B are the images that depict the physiological response 503A and can be compared to the images 402A.

Next, the light source 304 illuminates the retinal cells 308 with the light 310B after the image sensor 130 captures the images 402B, to cause the retinal cells 308 to exhibit a physiological response 503B (shown in FIG. 10). In some examples, the light source 304 illuminating the retinal cells 308 with the light 310B includes illuminating the retinal cells 308 with one or more (e.g., two or more) pulses with widths greater than 0.01 millisecond and less than 100 milliseconds. In various examples, the light 310B includes wavelengths greater than 380 nanometers and less than 780 nanometers.

As noted above, the illumination of the retinal cells 308 with the light 310B generally causes the retinal cells 308 to exhibit a second physiological response, an example of which is depicted in FIG. 10 as the physiological response 503B. In various examples, the physiological response 503B includes the retinal cells 308 exhibiting a change in shape and/or refractive index. For example, the physiological response 503B includes a contraction 505B during which the OPL of the retinal cells 308 decreases, followed by an expansion 506B during which the OPL increases. The contraction 505B can represent the retinal cells 308 physically shrinking and/or changing their biochemistry such that the refractive index of the retinal cells 308 decreases. The expansion 506B can represent the retinal cells 308 physically expanding and/or changing their biochemistry such that the refractive index of the retinal cells 308 increases. FIG. 10 depicts the respective time periods during which the contraction 505B and the expansion 506B occur and the changes in OPL.

The image sensor 130 also captures the images 402C of the retinal cells 308 after the light source 304 illuminates the retinal cells 308 with the light 310B. In some examples, capturing the images 402C includes capturing the images 402C in periodic succession after illuminating the retinal cells with the light 310B. The images 402C are the images that depict the physiological response 503B and can be compared to the images 402B.

The computing system 901 generates an output using the images 402A, the images 402B, and the images 402C. The output quantifies the physiological response 503A and the physiological response 503B and, in some examples, resembles the graph shown in FIG. 10. The computing system 901 generally writes the output to the computer readable medium 904 and/or presents the output on a display of the user interface 910.

In various examples, the retinal cells 308 can be illuminated sequentially with different wavelength ranges of light to generate the composite image 402E. Using this technique can achieve a resolution for the composite image 402E that exceeds the diffraction limit of the imaging light. Different retinal cells 308 generally have varying sensitivities to different wavelength ranges of visible light and such retinal cells 308 can be randomly dispersed within the retina. Thus, illuminating the retinal cells 308 with different wavelengths of light can help ensure that all of the retinal cells 308 are illuminated with light that is well tuned to generate a physiological response. The images 402B, the images 402C, and the images 402D can be mathematically combined (e.g., using thresholding and/or averaging) to generate the composite image 402E as described below.

After the images 402A, the images 402B, and the images 402C are captured, and after the retinal cells 308 are illuminated with the light 310A and the light 310B, the light source 304 illuminates the retinal cells 308 with the light 310D. In this example, the light 310A, the light 310B, and the light 310D have corresponding wavelength ranges that do not substantially overlap. For example, the light 310A could have wavelengths ranging from 400 to 450 nm, the light 310B could have wavelengths ranging from 450 nm to 550 nm, and the light 310D could have wavelengths ranging from 550 nm to 700 nm. After illumination with the light 310D, the image sensor 130 captures the images 402D of the retinal cells 308. The computing system 901 generates the composite image 402E of the retinal cells 308 using the images 402B, the images 402C, and the images 402D.

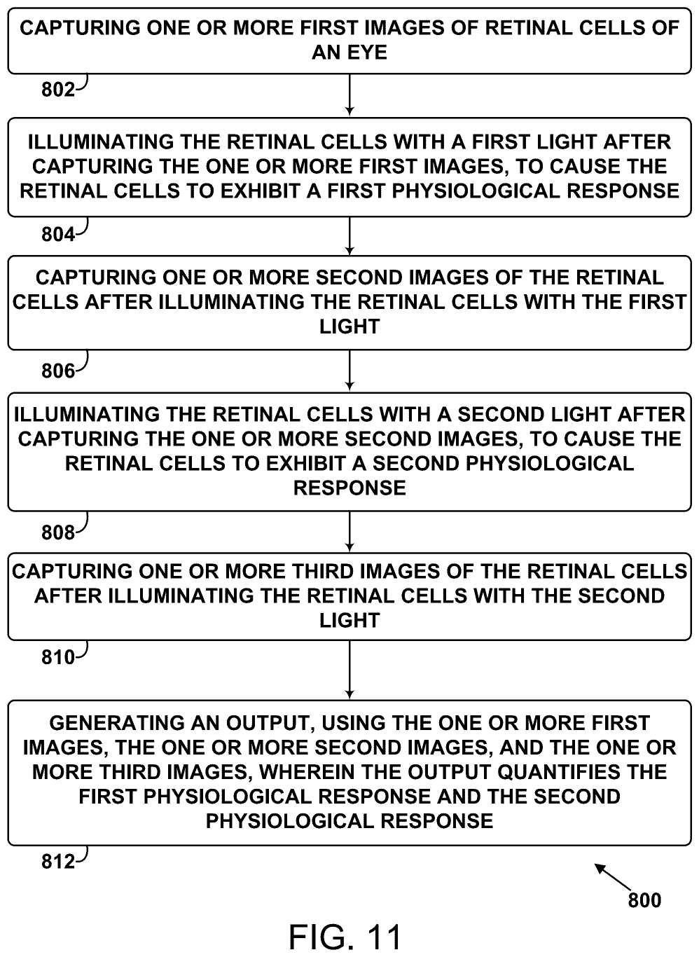

FIG. 11 is a block diagram of a method 800 of operating the imaging device 300. As shown in FIG. 11, the method 800 includes one or more operations, functions, or actions as illustrated by blocks 802, 804, 806, 808, 810, and 812. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 802, the method 800 includes the image sensor 130 capturing the images 402A of the retinal cells 308 of the eye 306. Functionality related to block 802 is described above with reference to FIGS. 8-10.

At block 804, the method 800 includes the light source 304 illuminating the retinal cells 308 with the light 310A after capturing the images 402A, to cause the retinal cells 308 to exhibit the physiological response 503A. Functionality related to block 804 is described above with reference to FIGS. 8-10.

At block 806, the method 800 includes the image sensor 130 capturing the images 402B of the retinal cells 308 after illuminating the retinal cells 308 with the light 310A. Functionality related to block 806 is described above with reference to FIGS. 8-10.

At block 808, the method 800 includes the light source 304 illuminating the retinal cells 308 with the light 310B after capturing the images 402B, to cause the retinal cells 308 to exhibit the physiological response 503B. Functionality related to block 808 is described above with reference to FIGS. 8-10.

At block 810, the method 800 includes the image sensor 130 capturing the images 402C of the retinal cells 308 after illuminating the retinal cells 308 with the light 310B. Functionality related to block 810 is described above with reference to FIGS. 8-10.

At block 812, the method 800 includes the computing system 901 generating the output, using the images 402A, the images 402B, and the images 402C. The output quantifies the physiological response 503A and the physiological response 503B. Functionality related to block 812 is described above with reference to FIGS. 8-10.

### Additional Examples

The optoretinogram (ORG) fills a much-needed gap in the objective, non-invasive, and sensitive assessment of retinal health. Photoreceptors and the outer retina in general are affected by various inherited and age-related macular degeneration. For treatments to mitigate these diseases, biomarkers for testing will need to be established and assessed comprehensively for their safety and efficacy. The existing methods fall short in either their resolution, sensitivity, or non-invasiveness. The ORG works by measuring tiny and fast (millisecond, nanometer) changes in the retina in response to a light stimulus and provides an unprecedented view into the function of retinal cells when healthy, diseased, or responding to treatment.

Manipulating the light stimulus properties unravels features of the ORG pertinent to retinal disease and management. Here, we detail the features of the stimulation paradigm that are novel for the application of ORG. Importantly, these stimulation paradigms are independent of the imaging technology used for capturing images of the retina. This independence is attributed to the observed utility of manipulating visual stimulation in revealing the inherent physiological properties of the retina. The cellular and molecular basis of the ORG can be established, and methods can be standardized for application in the clinical setting. The disclosed methods are independent of the choice of imaging instrument.

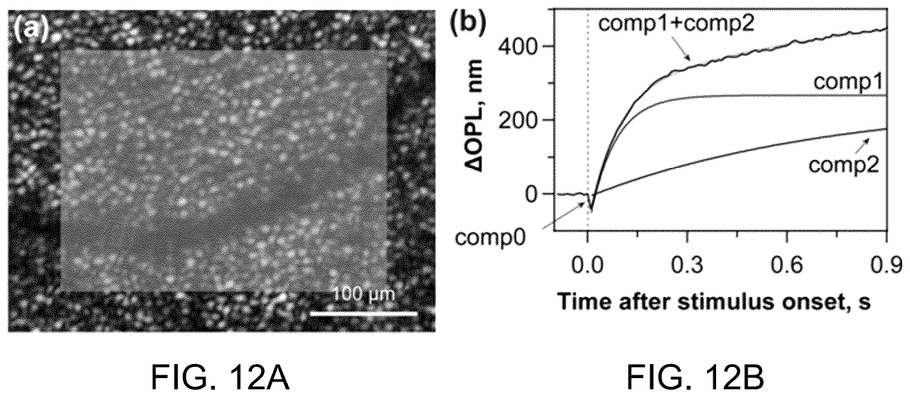

FIG. 12A is an image depicting optical phase change of an area of a retina. The basis of the visual stimulation methods is based on specific physiological properties of the retina. The human cone ORG can be decomposed into three additive components as shown in FIG. 12B: an initial shrinkage component (“Comp0”) and two elongation components (“Comp1”, “Comp2”), distinguishable by their characteristic sign (OS length increase or decrease), amplitude, light sensitivity kinetics and recovery. Comp0 and Comp2 can arise from biophysical consequences of photoisomerization while Comp1 of the ORG, with its substantially higher photosensitivity and faster kinetics compared to Comp2, can arise as an osmotic response to a highly amplified byproduct of phototransduction.

In retinal electrophysiology, manipulating the stimulus characteristics can be used to study how the different cells and circuits encode visual information. The stimulus variables are informed and refined in a closed loop as more information about their impact on retinal circuitry becomes available. A similar paradigm was non-existent in ORG until recently.

Wavelength spectrum: The photoreceptors lie at the very first stage of vision and have chromophores differently tuned to the visible spectrum. Furthermore, intrinsically photosensitive ganglion cells are also sensitive to specific wavelengths in the visible spectrum. Therefore, manipulating the center wavelength and spectrum is important to delineate the contribution of different photoreceptors and retinal ganglion cells to the ORG.

Multi-wavelength ORG: Retinal camera signals (OCT, SLO, fundus camera images, videos, set of volumes) are acquired with stimulus wavelengths spanning the visible spectrum from 400-700 nm. In addition, near-infrared wavelengths greater than 700 nm could be used that excite chromophores with 2-photon excitation, such that a wavelength of 1000 nm with high photon flux density (short, femtosecond or picosecond scale pulses of high energy) would effectively equal an excitation at 500 nm. This multiwavelength ORG can be used for the classification of cone spectral types. When used in conjunction with the temporal and spatial stimulation paradigms described below, multi-wavelength stimulation can selectively activate specific cone types more effectively than the single-flash, multi-wavelength condition.

Intensity & contrast: The light and contrast sensitivity (activity level vs. stimulus strength or contrast) of photoreceptors and their ensuing pathways are an important gauge of their functional activity. Previously, we have observed marked differences in the light sensitivity of rods and cones, and within the different ORG components in cones. Therefore, light intensity and contrast are key stimulus parameters under the realm of the ORG to probe the health of photoreceptors.

Dark-adapted ORG: Retinal camera signals are recorded with visual stimulation following dark adaptation ranging from 30 seconds to 30 mins or more. Shorter (30 seconds to 5 mins) and longer (30 mins) dark adaptation times are aimed at studying cones and rods respectively. The stimulus intensity is varied in accordance with the differential sensitivity of rods and cones. Rods have higher sensitivity and saturate at lower light intensity, while cones have lower sensitivity and saturate in their responses at higher light levels. Therefore, in the same retinal camera recording, a lower stimulus intensity activates rods, but does not activate cones. On the other hand, a higher stimulus intensity completely saturates rods and activates only cones. Thus, altering the stimulus intensity in a manner suited to their respective sensitivity allows differentiating rod and cone responses even without requiring their visualization on a cellular scale. The intensity of the stimulus can be varied by changing the power or pulse-width of the light flash. The range of intensities is broad, dictated on the lower end by the smallest measurable signal sensitivity of the retinal camera and on the higher end by the intensity where rods and cone response properties are completely saturated.

Light-adapted ORG: Retinal camera signals are recorded with visual stimulation following light adaptation. Light adaptation is implemented by varying the background light intensity (I) across a wide range. For a constant background, an increment or decrement (ΔI) stimulus of varying contrast is shown on the background (I). The contrast is increased to saturate the responses of cone photoreceptors.

Temporal properties: The different stages of retinal processing have specific temporal sensitivity and activation characteristics. In photoreceptors, the aforementioned ORG components have specific kinetics of activation and recovery, and these are mediated by the phototransduction amplification cascade and the visual cycle. Similarly, retinal ganglion cells have specific activity kinetics depending on their type. Varying the temporal properties of the stimulus provides access to these physiological phenomena in the inner and outer retina.

Single-flash, paired-flash, quad-flash, and serial flash ORG: The single-flash paradigm is the most basic allowing the inspection of the photosensitivity and kinetics of the components. The paired-flash paradigm can be used to study the recovery of different steps in the phototransduction cascade. Two flashes—test & probe—are used, with various inter-stimulus-intervals (ISIs) and a fixed test flash intensity. Depending on its energy density, the test flash suppresses or saturates the responses & reactions, while the 2^{nd} probe flash monitors the kinetics of recovery as a function of the ISI (see FIG. 10). We observed an apparent truncating intrusion by Comp1 during the Comp0 response, such that Comp1's fast velocity did not allow Comp0 to fully reveal its characteristics. The high photosensitivity of Comp1 suggested that an initial flash of relatively low intensity might suppress Comp1, enabling a second flash to more fully reveal the extent and kinetics of Comp0. This prediction was borne out and has led to major improvements in measurement of Comp0 amplitude compared to past efforts. Besides revealing the kinetics of Comp0, variants of the paired flash paradigm (e.g. quad flash or serial flash) are powerful tools for investigating the recovery of the ORG components, e.g. quantifying the bleached photopigment, comparing Comp0 and 2 recovery to pigment regeneration, and extracting new information about the enzymes and substrates underlying Comp1.

Single-Flash: Retinal camera signals are recorded upon a single stimulus illuminating the retina with a selected energy density, after a delay defined by the number of volumes.

Paired-Flash: Retinal camera signals are recorded with a pair of single-flash stimuli, that can have the same or different energy. A defined inter-stimulus interval (ISI) separates each flash. The rationale is explained above.

Quad-Flash: A quad-flash paradigm uses two paired-flash stimuli with the same or different ISI between each flash within a pair. The two pairs are separated by varying time intervals to study the time course of pigment regeneration. Retinal camera signals are recorded for the first and/or the second paired flash. The first pair serves to set the pigment concentration to a known starting point, obtained from the paired-flash paradigm.

Serial-Flash: Retinal camera signals are continuously recorded with multiple flash stimuli each with the same or different energy. The repeating duration, frequency and number of flashes can be varied.

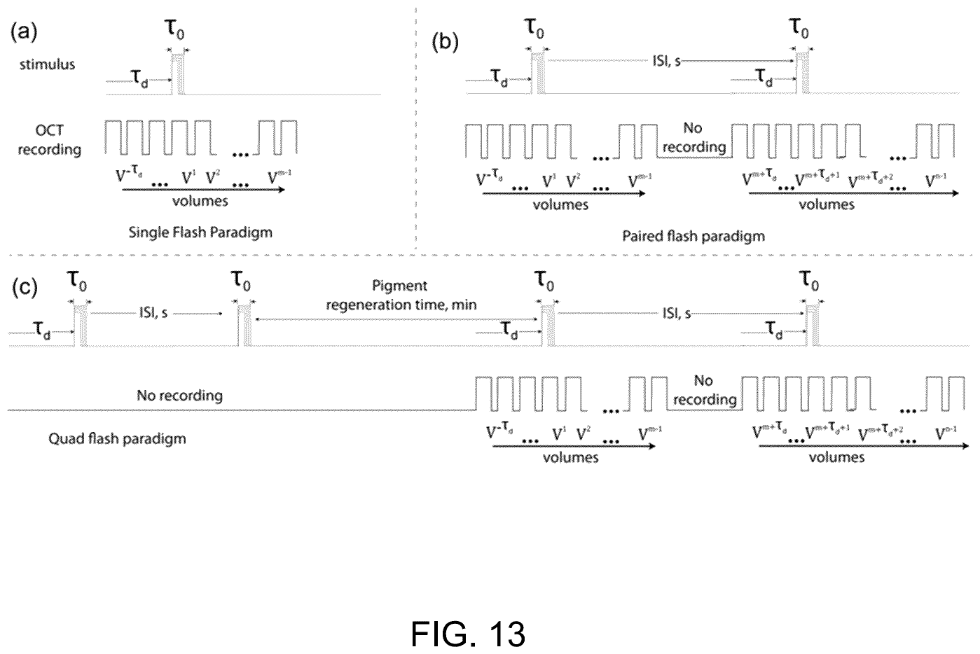

All four flash paradigms are depicted in FIG. 13. In all four paradigms, flash wavelength, intensity and contrast can be varied for any of the flashes. The time for dark adaptation can vary from 0.5 min to 5 min for cones, while for rods, the dark adaptation times can be up to 30 minutes or more, in order to assess photoreceptor specific pigment regeneration and visual cycle. Abnormalities in the visual cycle are one of the early biomarkers of age-related and inherited macular degenerations. These paradigms, as described above, pertain to full-field visual stimulation. However, the following spatial properties of the stimulus can be incorporated in addition.

Referring to FIG. 13, panel (a) corresponds to single-flash, (b) corresponds to paired-flash, and (c) corresponds to quad-flash. Time before stimulus onset is Ta, the pulse width of stimulus is to, and ISI represents interstimulus intervals. The serial flash paradigm is a generalized form of the paired flash, where number of pulses, frequency, and repeating duration can be varied.

Spatial properties: Retinal cell types have specific spatial arrangements. For example, cone photoreceptors are densely packed in the foveal center and there are no rods in the fovea. The size, density, spacing and arrangement of rods and cones change dramatically as a function of retinal eccentricity. This is also the case for retinal ganglion cells. Therefore, varying the spatial pattern of stimulation in a manner consistent with the cellular organization is important for delineating the contribution of different cell types to the ORG. Examples include:

Full-field ORG: Retinal camera signals are recorded upon a single full-field stimulus illuminating the retina. Any of the above stimulus properties can be varied for the full-field stimulus, for example different wavelengths, intensity, contrast, temporal flicker frequency, and number of pulses may be used.

Multi-focal and pattern ORG: Retinal camera signals are recorded upon a multi-focal or pattern stimulation, where multiple focal stimuli in a pattern could be distributed across the stimulus field such that each focal stimulus can be manipulated together or independently in any of the above dimensions-wavelength, intensity, contrast, temporal flicker frequency, and number of pulses. The size of each focal stimulus and its distribution across the retina may reflect the anatomy of the photoreceptors or retinal ganglion cells, such that the pattern has higher density and sampling in the fovea, decreasing with eccentricity.

Gaze-contingent stimulus: This is a case where the aforementioned stimulus moves in accordance with eye movements using an eye-tracking signal. The eye-tracking signal may be recovered from an image of eye's pupil and Purkinje reflections or from the retina camera image/volume stream.

Stimulus blur: The stimulus may be subjected to a pre-defined blur imposed by trial lenses or a wavefront corrector (deformable mirror, spatial light modulator). The distribution of the blur may be localized and variable or distributed uniformly across the retina. Both monochromatic and chromatic blur is possible.

Multiplexing stimulus variables: While individual variation in the aforementioned stimulus variables is informative, it must be emphasized that multiplexing these variables provides substantial benefits. Therefore, co-variation one or more of the above stimulus characteristics is an important addition to the visual stimulus protocols described. One specific example is:
Super-resolution ORG: The resolution of every optical imaging system is generally restricted by the diffraction-limit. This variation of ORG is aimed at overcoming this limit. A series of patterns or spatially coded stimuli are incident on the retina that selectively and sparsely activate a subset of cells, while a retinal camera records the elicited activity from these cells. With the prior activation, these cells are rendered less sensitive or inactive. When the next series of sparse activations impinge on the retina, a new subset of cells are activated, and this process continues until all cells are activated by and desensitized following the stimulus. The retinal camera recordings are then accumulated with time to generate a cumulative high-resolution image that surpasses the diffraction-limit. Under ordinary circumstances, when all cells are activated together, neighboring cells are blurred at the resolution limit imposed by diffraction. The super-resolution imaging protocol is prevalent in fluorescence microscopy with many variants such as PALM (Photoactivated localization microscopy) and STORM (Stochastic optical reconstruction microscopy). Its implementation can require knowing the activation, depletion and bleaching kinetics of fluorophores. An analogous application in the retina can require knowledge of the sensitivity and kinetics of the activated cell to the visual stimulus. This disclosure provides an avenue to translate super-resolution imaging methods to ORG in living eyes for the first time.

### Enumerated Example Embodiments (Eees)

EEE 1 is a method comprising: capturing one or more first images of retinal cells of an eye; illuminating the retinal cells with a first light after capturing the one or more first images, to cause the retinal cells to exhibit a first physiological response; capturing one or more second images of the retinal cells after illuminating the retinal cells with the first light; illuminating the retinal cells with a second light after capturing the one or more second images, to cause the retinal cells to exhibit a second physiological response; capturing one or more third images of the retinal cells after illuminating the retinal cells with the second light; and generating an output, using the one or more first images, the one or more second images, and the one or more third images, wherein the output quantifies the first physiological response and the second physiological response.

EEE 2 is the method of EEE 1, wherein illuminating the retinal cells with the first light comprises illuminating the retinal cells with the first light with a photon flux density of 10⁴ μm⁻² to 10⁸ μm⁻².

EEE 3 is the method of any one of EEEs 1-2, wherein the first physiological response comprises the retinal cells exhibiting a first change in shape and/or refractive index, and wherein the output indicates the first change in shape and/or refractive index and a first time period during which the first change in shape and/or refractive index occurs.

EEE 4 is the method of EEE 3, wherein the second physiological response comprises the retinal cells exhibiting a second change in shape and/or refractive index, and wherein the output indicates the second change in shape and/or refractive index and a second time period during which the second change in shape and/or refractive index occurs.

EEE 5 is the method of any one of EEEs 1-4, wherein the first physiological response comprises the retinal cells exhibiting a first contraction and then a first expansion, and wherein the output indicates a first length of the first contraction, a first time period during which the first contraction occurs, a second length of the first expansion, and a second time period during which the first expansion occurs.

EEE 6 is the method of EEE 5, wherein the second physiological response comprises the retinal cells exhibiting a second contraction and then a second expansion, and wherein the output indicates a third length of the second contraction, a third time period during which the second contraction occurs, a fourth length of the second expansion, and a fourth time period during which the second expansion occurs.

EEE 7 is the method of any one of EEEs 1-6, wherein capturing the one or more first images comprises capturing a plurality of images in periodic succession before illuminating the retinal cells with the first light.

EEE 8 is the method of any one of EEEs 1-7, wherein illuminating the retinal cells with the first light comprises illuminating the retinal cells with the first light having one or more pulses with widths greater than 0.01 millisecond and less than 100 milliseconds.

EEE 9 is the method of any one of EEEs 1-8, wherein illuminating the retinal cells with the first light comprises illuminating the retinal cells with the first light having at least two pulses with widths greater than 0.01 millisecond and less than 100 milliseconds.

EEE 10 is the method of any one of EEEs 1-9, wherein illuminating the retinal cells with the first light comprises illuminating the retinal cells with light having wavelengths greater than 380 nanometers and less than 780 nanometers.

EEE 11 is the method of any one of EEEs 1-10, wherein capturing the one or more second images comprises capturing a plurality of images in periodic succession after illuminating the retinal cells with the first light.

EEE 12 is the method of any one of EEEs 1-11, wherein illuminating the retinal cells with the second light comprises illuminating the retinal cells with the second light having one or more pulses with widths greater than 0.01 millisecond and less than 100 milliseconds.

EEE 13 is the method of any one of EEEs 1-12, wherein illuminating the retinal cells with the second light comprises illuminating the retinal cells with the second light having at least two pulses with widths greater than 0.1 millisecond and less than 100 milliseconds.

EEE 14 is the method of any one of EEEs 1-13, wherein illuminating the retinal cells with the second light comprises illuminating the retinal cells with light having wavelengths greater than 380 nanometers and less than 780 nanometers.

EEE 15 is the method of any one of EEEs 1-14, wherein capturing the one or more third images comprises capturing a plurality of images in periodic succession after illuminating the retinal cells with the second light.

EEE 16 is the method of any one of EEEs 1-15, wherein capturing the one or more first images, capturing the one or more second images, and capturing the one or more third images each comprises illuminating the retinal cells with a fourth light having wavelengths greater than 780 nanometers and detecting a fifth light emitted from the retinal cells in response to the fourth light, wherein the one or more first images, the one or more second images, and the one or more third images each indicate an intensity or a phase of the fifth light.

EEE 17 is the method of any one of EEEs 1-16, wherein capturing the one or more first images, capturing the one or more second images, and capturing the one or more third images each comprise using a scanning laser ophthalmoscope, a fundus camera, adaptive optics, or optical coherence tomography.

EEE 18 is the method of any one of EEEs 1-17, wherein illuminating the retinal cells with the first light comprises illuminating the retinal cells with the first light after obscuring the retinal cells from light for a time period of 0.1 seconds to 5 minutes.

EEE 19 is the method of EEE 18, wherein the first light has a photon flux density of at least 105 μm⁻².

EEE 20 is the method of any one of EEEs 1-19, wherein illuminating the retinal cells with the first light comprises illuminating the retinal cells with the first light after obscuring the retinal cells from light for a time period of 5 minutes to 30 minutes.

EEE 21 is the method of EEE 20, wherein the first light has a photon flux density of at least 104 μm⁻².

EEE 22 is the method of any one of EEEs 1-21, further comprising: illuminating the retinal cells with a third light prior to illuminating the retinal cells with the first light.

EEE 23 is the method of EEE 22, wherein the third light comprises wavelengths greater than 380 nanometers and less than 780 nanometers and a photon flux density of at least 104 μm⁻².

EEE 24 is the method of EEE 22, wherein the third light comprises wavelengths greater than 380 nanometers and less than 780 nanometers and a photon flux density of at least 105 μm⁻².

EEE 25 is the method of any one of EEEs 1-24, further comprising: illuminating the retinal cells with a third light after illuminating the retinal cells with the first light and with the second light, wherein exactly one of the first light, the second light, or the third light comprises wavelengths greater than 400 nanometers and less than 450 nanometers, wherein exactly one of the first light, the second light, or the third light comprises wavelengths greater than 450 nanometers and less than 550 nanometers, and wherein exactly one of the first light, the second light, or the third light comprises wavelengths greater than 550 nanometers and less than 700 nanometers, the method further comprising: capturing one or more fourth images of the retinal cells after illuminating the retinal cells with the third light; and generating a composite image of the retinal cells using the one or more second images, the one or more third images, and the one or more fourth images.

EEE 25 is the method of any one of EEEs 1-25, further comprising: illuminating the retinal cells with a third light after illuminating the retinal cells with the first light and with the second light, wherein the first light, the second light and the third light have corresponding wavelength ranges that do not substantially overlap, the method further comprising: capturing one or more fourth images of the retinal cells after illuminating the retinal cells with the third light; and generating a composite image of the retinal cells using the one or more second images, the one or more third images, and the one or more fourth images.

EEE 26 is a non-transitory computer readable medium storing instructions that, when executed by an imaging device, cause the imaging device to perform functions comprising: capturing one or more first images of retinal cells of an eye; illuminating the retinal cells with a first light after capturing the one or more first images, to cause the retinal cells to exhibit a first physiological response; capturing one or more second images of the retinal cells after illuminating the retinal cells with the first light; illuminating the retinal cells with a second light after capturing the one or more second images, to cause the retinal cells to exhibit a second physiological response; capturing one or more third images of the retinal cells after illuminating the retinal cells with the second light; and generating an output, using the one or more first images, the one or more second images, and the one or more third images, wherein the output quantifies the first physiological response and the second physiological response.

EEE 27 is an imaging device comprising: one or more processors; an image sensor; a light source; a user interface; and a computer readable medium storing instructions that, when executed by the one or more processors, cause the imaging device to perform functions comprising: capturing, using the image sensor, one or more first images of retinal cells of an eye; illuminating, using the light source, the retinal cells with a first light after capturing the one or more first images, to cause the retinal cells to exhibit a first physiological response; capturing, using the image sensor, one or more second images of the retinal cells after illuminating the retinal cells with the first light; illuminating, using the light source, the retinal cells with a second light after capturing the one or more second images, to cause the retinal cells to exhibit a second physiological response; capturing, using the image sensor, one or more third images of the retinal cells after illuminating the retinal cells with the second light; and generating an output via the user interface, using the one or more first images, the one or more second images, and the one or more third images, wherein the output quantifies the first physiological response and the second physiological response.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

What is claimed is:

1. A method comprising:
capturing one or more first images of retinal cells of an eye;
illuminating the retinal cells with a first light after capturing the one or more first images, to cause the retinal cells to exhibit a first physiological response;
capturing one or more second images of the retinal cells after illuminating the retinal cells with the first light;
illuminating the retinal cells with a second light after capturing the one or more second images, to cause the retinal cells to exhibit a second physiological response;
capturing one or more third images of the retinal cells after illuminating the retinal cells with the second light; and
generating an output, using the one or more first images, the one or more second images, and the one or more third images, wherein the output quantifies the first physiological response and the second physiological response.

2. The method of claim 1, wherein the first physiological response comprises the retinal cells exhibiting a first change in shape and/or refractive index, and wherein the output indicates the first change in shape and/or refractive index and a first time period during which the first change in shape and/or refractive index occurs.

3. The method of claim 2, wherein the second physiological response comprises the retinal cells exhibiting a second change in shape and/or refractive index, and wherein the output indicates the second change in shape and/or refractive index and a second time period during which the second change in shape and/or refractive index occurs.

4. The method of claim 1, wherein the first physiological response comprises the retinal cells exhibiting a first contraction and then a first expansion, and wherein the output indicates a first length of the first contraction, a first time period during which the first contraction occurs, a second length of the first expansion, and a second time period during which the first expansion occurs.

5. The method of claim 4, wherein the second physiological response comprises the retinal cells exhibiting a second contraction and then a second expansion, and wherein the output indicates a third length of the second contraction, a third time period during which the second contraction occurs, a fourth length of the second expansion, and a fourth time period during which the second expansion occurs.

6. The method of claim 1, wherein capturing the one or more first images comprises capturing a plurality of images in periodic succession before illuminating the retinal cells with the first light.

7. The method of claim 1, wherein illuminating the retinal cells with the first light comprises illuminating the retinal cells with the first light having one or more pulses with widths greater than 0.01 millisecond and less than 100 milliseconds.

8. The method of claim 1, wherein illuminating the retinal cells with the first light comprises illuminating the retinal cells with the first light having at least two pulses with widths greater than 0.01 millisecond and less than 100 milliseconds.

9. The method of claim 1, wherein illuminating the retinal cells with the first light comprises illuminating the retinal cells with light having wavelengths greater than 380 nanometers and less than 780 nanometers.

10. The method of claim 1, wherein capturing the one or more second images comprises capturing a plurality of images in periodic succession after illuminating the retinal cells with the first light.

11. The method of claim 1, wherein illuminating the retinal cells with the second light comprises illuminating the retinal cells with the second light having one or more pulses with widths greater than 0.01 millisecond and less than 100 milliseconds.

12. The method of claim 1, wherein illuminating the retinal cells with the second light comprises illuminating the retinal cells with the second light having at least two pulses with widths greater than 0.1 millisecond and less than 100 milliseconds.

13. The method of claim 1, wherein capturing the one or more third images comprises capturing a plurality of images in periodic succession after illuminating the retinal cells with the second light.

14. The method of claim 1, wherein capturing the one or more first images, capturing the one or more second images, and capturing the one or more third images each comprises illuminating the retinal cells with a fourth light having wavelengths greater than 780 nanometers and detecting a fifth light emitted from the retinal cells in response to the fourth light,
wherein the one or more first images, the one or more second images, and the one or more third images each indicate an intensity or a phase of the fifth light.

15. The method of claim 1, wherein illuminating the retinal cells with the first light comprises illuminating the retinal cells with the first light after obscuring the retinal cells from light for a time period of 0.1 seconds to 5 minutes.

16. The method of claim 1, wherein illuminating the retinal cells with the first light comprises illuminating the retinal cells with the first light after obscuring the retinal cells from light for a time period of 5 minutes to 30 minutes.

17. The method of claim 1, further comprising: illuminating the retinal cells with a third light prior to illuminating the retinal cells with the first light, wherein the third light comprises wavelengths greater than 380 nanometers and less than 780 nanometers and a photon flux density of at least 104 μm⁻².

18. The method of claim 1, further comprising:
illuminating the retinal cells with a third light after illuminating the retinal cells with the first light and with the second light, wherein the first light, the second light and the third light have corresponding wavelength ranges that do not substantially overlap, the method further comprising:
capturing one or more fourth images of the retinal cells after illuminating the retinal cells with the third light; and
generating a composite image of the retinal cells using the one or more second images, the one or more third images, and the one or more fourth images.

19. A non-transitory computer readable medium storing instructions that, when executed by an imaging device, cause the imaging device to perform functions comprising:
capturing one or more first images of retinal cells of an eye;
illuminating the retinal cells with a first light after capturing the one or more first images, to cause the retinal cells to exhibit a first physiological response;
capturing one or more second images of the retinal cells after illuminating the retinal cells with the first light;
illuminating the retinal cells with a second light after capturing the one or more second images, to cause the retinal cells to exhibit a second physiological response;
capturing one or more third images of the retinal cells after illuminating the retinal cells with the second light; and
generating an output, using the one or more first images, the one or more second images, and the one or more third images, wherein the output quantifies the first physiological response and the second physiological response.

20. An imaging device comprising:
one or more processors;
an image sensor;
a light source;
a user interface; and
a computer readable medium storing instructions that, when executed by the one or more processors, cause the imaging device to perform functions comprising:
capturing, using the image sensor, one or more first images of retinal cells of an eye;
illuminating, using the light source, the retinal cells with a first light after capturing the one or more first images, to cause the retinal cells to exhibit a first physiological response;
capturing, using the image sensor, one or more second images of the retinal cells after illuminating the retinal cells with the first light;
illuminating, using the light source, the retinal cells with a second light after capturing the one or more second images, to cause the retinal cells to exhibit a second physiological response;
capturing, using the image sensor, one or more third images of the retinal cells after illuminating the retinal cells with the second light; and
generating an output via the user interface, using the one or more first images, the one or more second images, and the one or more third images, wherein the output quantifies the first physiological response and the second physiological response.
